(19) United States
(12) Patent Application Publication
(10) Pub. No.: US 2026/0174334 A1
(43) Pub. Date: Jun. 25, 2026

(54) LIVING BODY DETECTION APPARATUS

(71) Applicant: SEIKO EPSON CORPORATION, Tokyo (JP)

(72) Inventor: Hiromu TAKAYAMA, CHINO-SHI (JP)

(73) Assignee: SEIKO EPSON CORPORATION, Tokyo (JP)

(21) Appl. No.: 19/430,331

(22) Filed: Dec. 23, 2025

(30) Foreign Application Priority Data
Dec. 25, 2024 (JP) 2024-228157

(51) Int. Cl. A61B 5/00

(52) U.S. Cl. CPC A61B 5/0059

(57) ABSTRACT

A living body detection apparatus includes a light emitter that emits first light, a first reflector that reflects the first light toward a detection target living body, a light receiver that receives second light reflected from the detection target living body on which the first light is incident, a detector that detects living body information on the detection target living body based on the second light, and a first substrate that is disposed between the light emitter and the light receiver and blocks the first light. The light emitter is disposed between the light receiver and the first reflector.

The present application is based on, and claims priority from JP Application Serial Number 2024-228157, filed December 25, 2024, the disclosure of which is hereby incorporated by reference herein in its entirety.

### BACKGROUND

### 1. Technical Field

The present disclosure relates to a living body detection apparatus, for example, an apparatus that can be preferably used to optically detect living body information.

### 2. Related Art

JP-A-2009-106373 discloses an apparatus that senses surface layer tissue of a living body. The apparatus that senses surface layer tissue of a living body disclosed in JP-A-2009-106373 senses surface layer tissue of a living body with light emitted from an end-surface-emission-type light emitter that emits light via the entire circumferential end surface thereof, and receives diffusively reflected light from the surface layer tissue of the living body with a surface-incidence-type light receiver. The light receiver is layered on the light emitter.

JP-A-2009-106373 is an example of the related art.

In the configuration of the apparatus that senses surface layer tissue of a living body according to JP-A-2009-106373, to provide multiple light emitters that emit light beams having multiple colors, it is necessary to stack the multiple light emitters on each other under the light receiver, so that there is no prospect of further reduction in size of the entire apparatus that senses surface layer tissue of a living body.

### SUMMARY

Numbers and characters used in the embodiments of the present disclosure are used to describe configurations for solving the challenges. The numbers and characters are added in parentheses for reference to show an example of the correspondence between the description of the claims and the embodiments of the disclosure. The claims therefore should not be construed as being limited by the description in parentheses.

According to an embodiment, a living body detection apparatus (1) includes a light emitter (3), a first reflector (41), a light receiver (5), a first substrate (22), and a detector (72). The light emitter (3) emits first light (81). The first reflector (41) reflects the first light (81) in a first direction (+Z direction). When a detection target living body (9) is present in the first direction (+Z direction) as viewed from the first reflector (41), the light receiver (5) receives second light (82) that is the first light (81) reflected from the detection target living body (9). The first substrate (22) is disposed between the light emitter (3) and the light receiver (5), and blocks the first light (81). The detector (72) detects living body information on the detection target living body (9) based on the second light (82). In a plan view viewed from the detection target living body (9), the light emitter (3) is disposed between the light receiver (5) and the first reflector (41). The light emitter (3) emits the first light (81) toward the first reflector (41).

### BRIEF DESCRIPTION OF THE DRAWINGS

FIG. 1 is a cross-sectional view of an example of the configuration of a living body detection apparatus according to a first embodiment as viewed from a first direction.

FIG. 2 is a plan view of the example of the configuration of the living body detection apparatus shown in FIG. 1 as viewed from a second direction perpendicular to the first direction.

FIG. 3 is a block circuit diagram schematically showing an example of the electrical coupling in the living body detection apparatus according to the first embodiment.

FIG. 4 is a cross-sectional view of an example of the configuration of a living body detection apparatus according to a second embodiment as viewed from the first direction.

FIG. 5 is a plan view of the example of the configuration of the living body detection apparatus shown in FIG. 4 as viewed from the second direction perpendicular to the first direction.

FIG. 6 is a cross-sectional view of an example of the configuration of a living body detection apparatus according to a third embodiment as viewed from the first direction.

FIG. 7 is a plan view of the example of the configuration of the living body detection apparatus shown in FIG. 6 as viewed from the second direction perpendicular to the first direction.

FIG. 8 is a block circuit diagram schematically showing an example of the electrical coupling in the living body detection apparatus according to the third embodiment.

### DESCRIPTION OF EMBODIMENTS

Embodiments of a living body detection apparatus according to the present disclosure will be described below with reference to the accompanying drawings.

### First embodiment

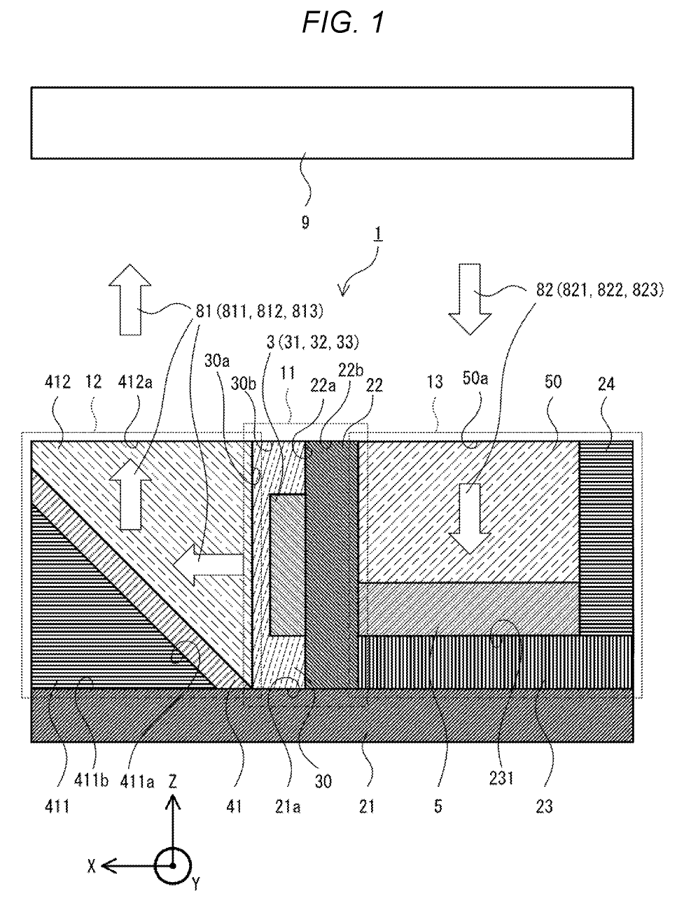

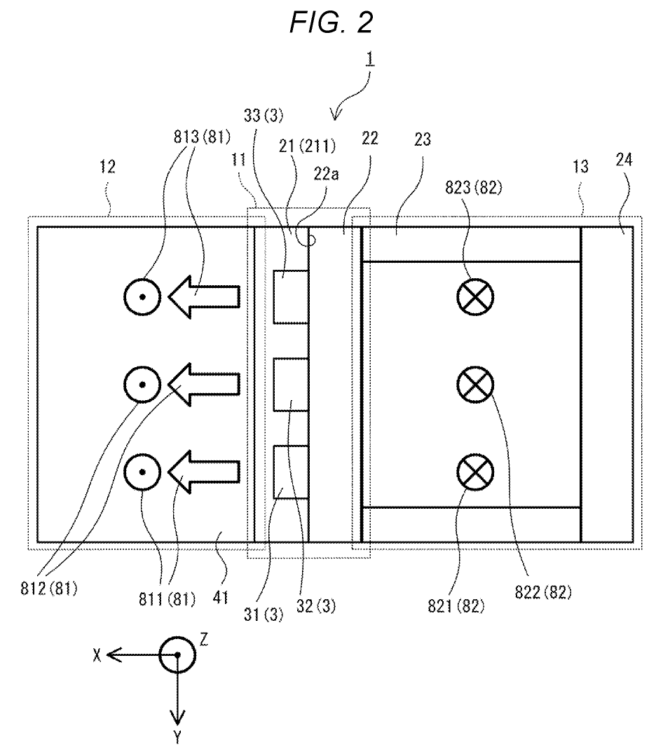

FIG. 1 is a cross-sectional view of an example of the configuration of a living body detection apparatus 1 according to a first embodiment as viewed from a first direction (Y direction). FIG. 2 is a plan view of the example of the configuration of the living body detection apparatus 1 shown in FIG. 1 as viewed from a second direction (Z direction) perpendicular to the first direction. The living body detection apparatus 1 according to the embodiment includes a first substrate 21, and a first unit 11, a second unit 12, and a third unit 13 mounted on a mounting surface 21a of the first substrate 21, as shown in FIGS. 1 and 2. In FIGS. 1 and 2, broken line frames representing the first unit 11, the second unit 12, and the third unit 13 partially overlap with each other because they are expanded for clarity, and elements contained in each of the first unit 11, the second unit 12, and the third unit 13 are described below. The living body detection apparatus 1 is an apparatus that detects living body information on a detection target living body 9 by using an optical, non-invasive method. Examples of the detection target living body 9 may include a human or an animal. Examples of the living body information may include the pulse and the blood oxygen saturation (blood oxygen concentration). The living body detection apparatus 1 is, for example, a photo-plethysmography (PPG) sensor, and is incorporated, for example, in a vital instrument.

The first unit 11 includes a second substrate 22, a light emitter 3 mounted on a first mounting surface 22a of the second substrate 22, and a protector 30, which protects the light emitter 3. The light emitter 3 includes a first light emitter 31, which outputs output light 811 having a first color, a second light emitter 32, which outputs output light 812 having a second color, and a third light emitter 33, which outputs output light 813 having a third color. The first light emitter 31, the second light emitter 32, and the third light emitter 33 may each be a light emitting diode (LED), an organic light emitting diode (OLED), a micro-LED, or a vertical cavity surface emitting laser (VCSEL). The first light emitter 31, the second light emitter 32, and the third light emitter 33 may each output the output light 811, 812, and 813 in the form of surface emission.

As an example, the first color of the first output light 811 is green, which is suitable for pulse measurement, and the wavelength of the first output light 811 may fall within a band of about 520 nm (nanometers). The second color of the second output light 812 and the third color of the third output light 813 are red and infrared, which are suitable for blood oxygen saturation concentration measurement, and the wavelengths of the second output light 812 and the third output light 813 may fall within the bands of 660 nm and 905 nm. In the following description, when the first output light 811, the second output light 812, and the third output light 813 are not distinguished from each other, the output light 811, the output light 812, and the output light 813 are collectively referred to as output light 81 in some cases.

The second substrate 22 is disposed so as to extend in a second planar direction (YZ direction) that intersects with a first planar direction (XY direction) in which the first substrate 21 extends. As an example, the second substrate 22 is so disposed that the first mounting surface 22a of the second substrate 22 extends in the second plane direction (YZ direction) perpendicular to the first plane direction (XY direction), in which the mounting surface 21a of the first substrate 21 extends. As an example, the first light emitter 31, the second light emitter 32, and the third light emitter 33 emit the first output light 811, the second output light 812, and the third output light 813, respectively, in a third direction (+X direction) perpendicular to the first mounting surface 22a of the second substrate 22, on which the first light emitter 31, the second light emitter 32, and the third light emitter 33 are mounted. Note, however, that the output direction of the first output light 811, the second output light 812, and the third output light 813 may fall within a predetermined angular range within which the third direction falls.

The protector 30 may be made, for example, of transparent resin so as not to hinder the propagation of the output light 81. A surface 30a of the protector 30 is preferably in close contact with the surface of a protector 412 of the second unit 12 so as not to hinder the propagation of the output light 81. An end surface 30b of the protector 30 is preferably flush with an end surface 22b of the second substrate 22.

The second unit 12 includes a first reflector 41, which reflects the output light 81 from the light emitter 3 in a desired direction, a first support 411, which supports the first reflector 41, and the protector 412, which protects the surface of the first reflector 41. In FIG. 2, however, the first support 411 is hidden behind the first reflector 41, so that the protector 412 is not shown. The first reflector 41 reflects the output light 81, which is output by the light emitter 3 toward the first reflector 41 shifted in the third direction (+X direction), in the second direction (+Z direction), in which the detection target living body 9 is expected to be present when viewed from the living body detection apparatus 1. The first reflector 41 may be a mirror or a metal plate disposed on an inclined surface 411a of the first support 411, or may be a reflection layer formed at the inclined surface 411a of the first support 411 by photolithography, electron beam lithography, nanoimprinting, or any other method.

The reflectance at which the first reflector 41 reflects the output light 81 is preferably 100%, but the value of the reflectance is merely an example and does not limit the present disclosure. In the example shown in FIG. 1, the direction in which the light emitter 3 outputs the output light 81 (+X direction) is perpendicular to the propagation direction (+Z direction) of the output light 81 reflected from the first reflector 41. In the case described above, the angle between the reflection surface of the first reflector 41 and the direction in which the output light 81 is output from the light emitter 3 (+X direction) may be 45 degrees. The value of the angle is, however, merely an example and does not limit the present disclosure, and may be determined as appropriate based on an expected position of the detection target living body 9 with respect to the living body detection apparatus 1.

The first support 411 has the inclined surface 411a, which supports the first reflector 41, and a bottom surface 411b, which is in contact with the mounting surface 21a of the first substrate 21. The first support 411 may be made, for example, of black resin so as to absorb, when part of the output light 81 passes through the first reflector 41, the output light 81 having passed through the first reflector 41. The first support 411 may have a cavity therein.

The protector 412 protects a surface of the first reflector 41 that is the surface opposite the first support 411. As an example, the protector 412 may be made, for example, of transparent resin so as not to hinder the propagation of the output light 81. A surface 412a of the protector 412, which is a surface closest to the detection target living body 9, that is, a surface farthest from the first substrate 21, is preferably flush with the end surface 30b of the protector 30 of the first unit 11.

The third unit 13 includes a third substrate 23, a light receiver 5 mounted on a mounting surface 231 of the third substrate 23, a protector 50, which protects the surface of the light receiver 5, and a light-blocking wall 24. The light receiver 5 receives light having passed through the space between the second substrate 22 and the light-blocking wall 24 out of reflected light 82, which is the output light 81 reflected from the surface or the interior of the detection target living body 9. In more detail, the light receiver 5 receives first reflected light 821, second reflected light 822, and third reflected light 823, which are the first output light 811, the second output light 812, and the third output light 813 contained in the output light 81 and reflected from the surface of the detection target living body 9, respectively.

The second substrate 22 and the light-blocking wall 24 are disposed so as to prevent noise light other than the reflected light 82 from reaching the light receiver 5. The noise light includes light not reflected from the surface of the detection target living body 9 out of the output light 81 output by the light emitter 3, light further reflected from surfaces other than the detection target living body 9 out of the reflected light 82, which is the output light 81 reflected from the surface of the detection target living body 9. To prevent the output light 81 not having traveled via the detection target living body 9 from reaching the light receiver 5, the direction in which the light emitter 3 outputs the output light 81 (+X direction) is preferably opposite the direction in which the third unit 13 including the light receiver 5 is shifted (−X direction) from the first unit 11 including the light emitter 3. The second substrate 22 is disposed between the light emitter 3 and the light receiver 5, and the light emitter 3 is disposed between the first reflector 41 and the light receiver 5. The light-blocking wall 24 may be made, for example, of highly reflective resin such as white resin, or may be configured, for example, with a metal plate.

The protector 50 protects a surface of the light receiver 5 that is the surface opposite the third substrate 23. As an example, the protector 50 may be made, for example, of transparent resin so as not to hinder the propagation of the reflected light 82. A surface 50a of the protector 50, which is a surface closest to the detection target living body 9, that is, a surface farthest from the mounting surface 21a of the first substrate 21, may be flush with the end surface 22b of the second substrate 22, which is a surface closest to the detection target living body 9. The protector 50 is preferably thin so that the reflected light 82 from the detection target living body 9 is readily received by the light receiver 5. When there is a bonding wire that couples the light receiver 5 to the third substrate 23, however, the protector 50 is preferably thick enough to fix and protect the bonding wire.

As an example, the first unit 11, the second unit 12, and the third unit 13 may be produced independently of each other, and then bonded to the mounting surface 21a of the first substrate 21. When the living body detection apparatus 1 according to the present disclosure is produced as described above, it is expected, for example, that the degree of difficulty in producing the apparatus is reduced, the production yield is improved, and the production cost is reduced.

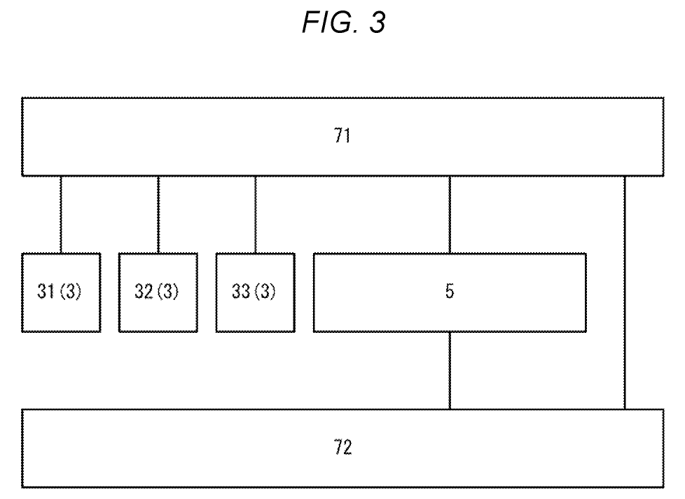

The living body detection apparatus 1 according to the embodiment further includes a controller 71, which controls the operations of the light emitter 3 and the light receiver 5, and a detector 72, which detects living body information on the detection target living body 9 based on the reflected light 82 received by the light receiver 5, as shown in FIG. 3. Each of the controller 71 and the detector 72 may be a functional block that realizes desired processing by an arithmetic operation unit and a storage unit of what is called a computer cooperating with each other to execute a predetermined program, or may be an electronic circuit that is incorporated in a large-scale integrated circuit or the like and realizes desired processing.

The controller 71 is electrically coupled to each of the first light emitter 31, the second light emitter 32, and the third light emitter 33 contained in the light emitter 3, and the light receiver 5. The electrical coupling between the controller 71 and the first light emitter 31, the second light emitter 32, and the third light emitter 33 may be realized by wiring provided on the first substrate 21 and/or the second substrate 22, or may be realized by bonding wires provided between the first light emitter 31, the second light emitter 32, and the third light emitter 33 and the second substrate 22.

The electrical coupling between the controller 71 and the light receiver 5 may be realized by wiring provided on the first substrate 21 and/or the third substrate 23, or may be realized by bonding wires provided between the light receiver 5 and the third substrate 23. Similarly, the electrical coupling between the detector 72 and the light receiver 5 may be realized by the wiring provided on the first substrate 21 and/or the third substrate 23, or may be realized by the bonding wires provided between the light receiver 5 and the third substrate 23.

The controller 71 controls the operations of the first light emitter 31, the second light emitter 32, the third light emitter 33, and the light receiver 5 by using electric control signals. The controller 71 may operate only some of the first light emitter 31, the second light emitter 32, the third light emitter 33, and the light receiver 5 at a time, or may operate all of them at a time. The detector 72 may operate constantly, or may operate only when the light receiver 5 operates. Switching between the state in which the detector 72 is in operation and the state in which the detector 72 is not in operation may be performed under the control of the controller 71.

As an example, in a first state in which the pulse is measured, the controller 71 causes the first light emitter 31 and the detector 72 to operate, and the second light emitter 32 and the third light emitter 33 not to operate. In this process, the light receiver 5 receives the first reflected light 821 derived from the first output light 811 output by the first light emitter 31. Since the light receiver 5 does not receive the second reflected light 822 or the third reflected light 823, which becomes noise when receiving the first reflected light 821, the detector 72 can accurately detect living body information on the detection target living body 9, that is, can accurately measure the pulse.

Similarly, in a second state in which the blood oxygen saturation concentration is measured, the controller 71 causes the second light emitter 32, the third light emitter 33, and the detector 72 to operate, and the first light emitter 31 not to operate. In this process, the light receiver 5 receives the second reflected light 822 and the third reflected light 823 derived from the second output light 812 and the third output light 813 output by the second light emitter 32 and the third light emitter 33. Since the light receiver 5 does not receive the first reflected light 821, which becomes noise when receiving the second reflected light 822 and the third reflected light 823, the detector 72 can accurately detect living body information on the detection target living body 9, that is, can accurately measure the blood oxygen saturation concentration.

Furthermore, the power consumed by the living body detection apparatus 1 according to the first embodiment of the present disclosure can be saved by controlling the multiple light emitters 31, 32, and 33 to perform the time-division operation.

An example of the operation of the living body detection apparatus 1 according to an embodiment will be described. The controller 71 first generates a control signal and transmits the control signal to the light emitter 3. In this process, the controller 71 may generate a first control signal, a second control signal, and a third control signal used to control the first light emitter 31, the second light emitter 32, and the third light emitter 33, respectively, and transmit the first to third control signals to the first light emitter 31, the second light emitter 32, and the third light emitter 33, respectively.

The light emitter 3 generates the output light 81 as first light in accordance with the received control signal, and outputs the output light 81. In more detail, the first light emitter 31 outputs the first output light 811 in accordance with the first control signal, the second light emitter 32 outputs the second output light 812 in accordance with the second control signal, and the third light emitter 33 outputs the third output light 813 in accordance with the third control signal. Only some of the first light emitter 31, the second light emitter 32, and the third light emitter 33 of the light emitter 3 may output the output light 81 at any point in time.

The output light 81 from the light emitter 3 passes through the protector 30 of the first unit 11 and the protector 412 of the second unit 12, is reflected from the first reflector 41, and exits the living body detection apparatus 1 via the surface 412a of the protector 412. When the detection target living body 9 is not present at the destination toward which the output light 81 is output, the light receiver 5 receives no light, and the detector 72 detects no living body information on the detection target living body 9. When the detection target living body 9 is present at the destination toward which the output light 81 is output, the output light 81 is reflected from the surface or the interior of the detection target living body 9. The output light 81 reflected from the surface or the interior of the detection target living body 9 is hereinafter referred to as the reflected light 82. When the output light 81 contains the first output light 811, the reflected light 82 contains the first reflected light 821. Similarly, when the output light 81 contains the second output light 812, the reflected light 82 contains the second reflected light 822, and when the output light 81 contains the third output light 813, the reflected light 82 contains the third reflected light 823. At least part of the reflected light 82 enters the protector 50 of the third unit 13. The light receiver 5 receives at least part of the reflected light 82 having entered the protector 50.

The light receiver 5 generates an electric signal corresponding to the characteristics of the received reflected light 82, and transmits the electric signal to the detector 72. The detector 72 detects the living body information on the detection target living body 9 based on the received electric signal. In more detail, when the reflected light 82 received by the light receiver 5 contains the first reflected light 821 derived from the first output light 811, the detector 72 may measure the pulse of the detection target living body 9 based on the received electric signal. When the reflected light 82 received by the light receiver 5 contains the second reflected light 822 and/or the third reflected light 823 derived from the second output light 812 and/or the third output light 813, the detector 72 may measure the blood oxygen saturation concentration of the detection target living body 9 based on the received electric signal.

As described above, in the living body detection apparatus 1 according to the embodiment, it is unnecessary to stack the multiple light emitters 31, 32, and 33 in the thickness direction (Z direction), which output the output light 811, 812, and 813 having different wavelengths, respectively, so that the reduction in the size of the apparatus can be expected, unlike in JP-A-2009-106373. In addition, since the second substrate 22, on which the light emitter 3 is mounted, prevents the output light 81 from directly reaching the light receiver 5, the noise light in the light receiver 5 is reduced, as compared with the configuration described in JP-A-2009-106373, so that living body information on the detection target living body 9 can be detected more accurately.

### Second embodiment

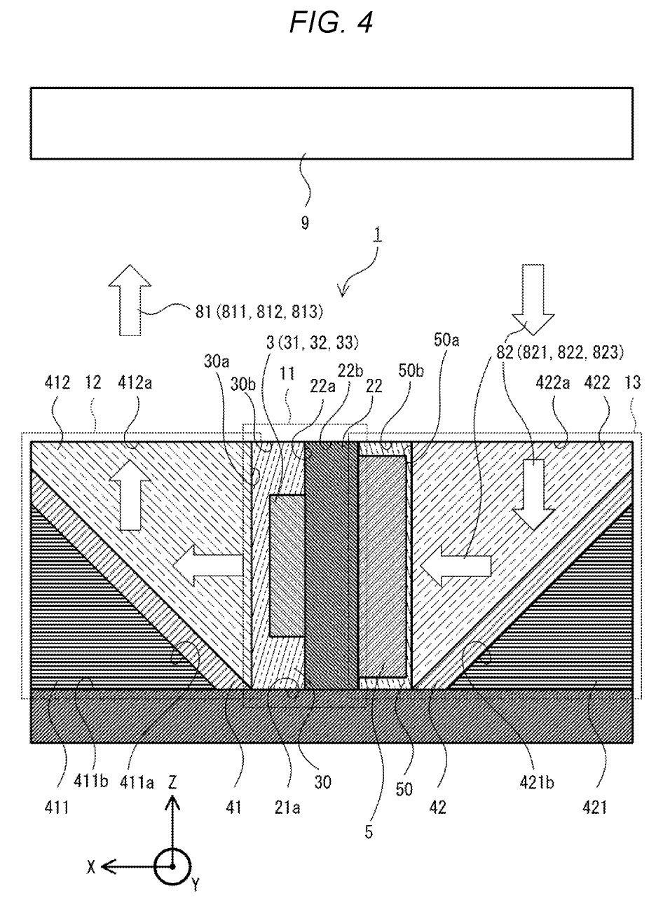

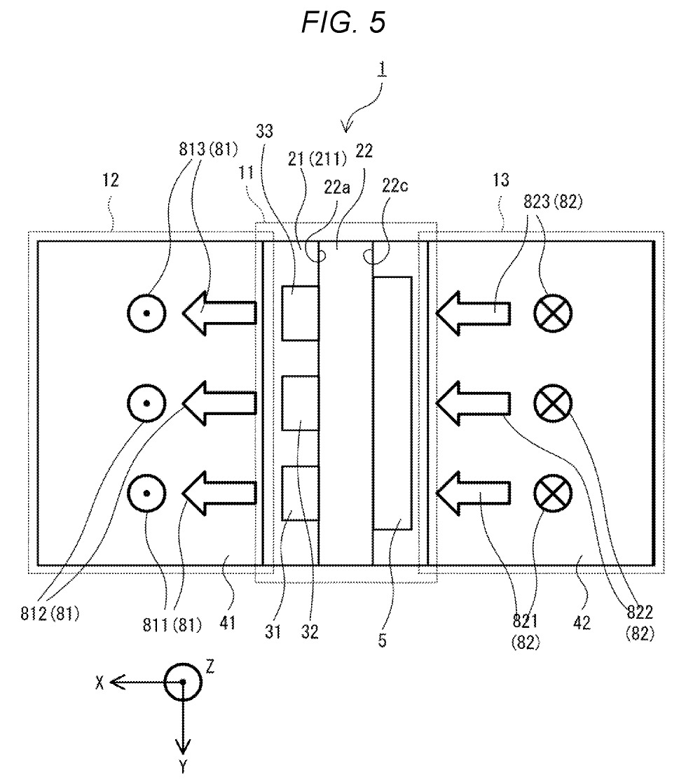

FIG. 4 is a cross-sectional view of an example of the configuration of a living body detection apparatus 1 according to a second embodiment as viewed from the first direction (Y direction). FIG. 5 is a plan view of the example of the configuration of the living body detection apparatus 1 shown in FIG. 4 as viewed from the second direction (Z direction) perpendicular to the first direction. The living body detection apparatus 1 according to the second embodiment is the living body detection apparatus 1 according to the first embodiment to which the following changes are made, as shown in FIGS. 4 and 5. Note in the second embodiment that portions common to those in the first embodiment will not be described in detail in some cases.

In the second unit 13, the light receiver 5 is mounted on a second mounting surface 22c of the second substrate 22, which is the surface facing the first mounting surface 22a. The planar direction (YZ direction) in which the second mounting surface 22c of the second substrate 22 extends is perpendicular to the output direction in which the light emitter 3 outputs the output light 81 (+X direction), and the light receiver 5 receives the reflected light 82 propagating in the output direction (+X direction) from the opposite side (−X side).

In the first unit 11, the light receiver 5 is protected by the protector 50 made, for example, of transparent resin. Out of the surfaces of the light receiver 5, the surface other than the surface facing the second substrate 22 may be covered with the protector 50. An end surface 50b of the protector 50, which is a surface closest to the detection target living body 9, that is, a surface farthest from the first substrate 21, is preferably flush with the end surface 22b of the second substrate 22.

The configurations of the first substrate 21 and the second unit 12 according to the second embodiment are the same as those in the first embodiment.

The third unit 13 according to the second embodiment includes a second reflector 42, a second support 421, which supports the second reflector 42, and a protector 422, which protects the surface of the second reflector 42, similarly to the first unit 11 according to the first embodiment. The second reflector 42, however, reflects the reflected light 82 from the detection target living body 9 toward the light receiver 5. In the example shown in FIG. 4, the direction in which the reflected light 82 from the detection target living body 9 propagates (−Z direction) is perpendicular to the direction in which the reflected light 82 reflected from the second reflector 42 propagates (+X direction). In this case, the angle between the reflection surface of the second reflector 42 and the propagation direction of the reflected light 82 toward the light receiver 5 (+X direction) may be 45 degrees. The value of the angle is, however, merely an example and does not limit the present disclosure, and may be determined as appropriate based on an expected position of the detection target living body 9 with respect to the living body detection apparatus 1.

Other configurations and characteristics of the second reflector 42 are the same as those of the first reflector 41. Other configurations of the third unit 13 according to the second embodiment are the same as those of the second unit 12 according to the first embodiment except that the direction parallel to the output direction of the output light 81 (+X direction) is reversed.

The configurations and operations of the controller 71 and the detector 72 according to the second embodiment are the same as those in the first embodiment. The operation of the living body detection apparatus 1 according to the second embodiment is also the same as that in the first embodiment.

As described above, in the second embodiment, the light emitter 3 and the light receiver 5 are mounted on the first mounting surface 22a and the second mounting surface 22c of the same second substrate 22, which face each other, respectively. As a result, also in the second embodiment, the second substrate 22 is disposed so as to prevent the output light 81 output by the light emitter 3 from directly reaching the light receiver 5. Furthermore, the third substrate 23 and the light-blocking wall 24 used in the first embodiment can be omitted, so that further reduction in size and further reduction in production cost can be expected in the living body detection apparatus 1 according to the second embodiment as compared with those in the first embodiment.

### Third embodiment

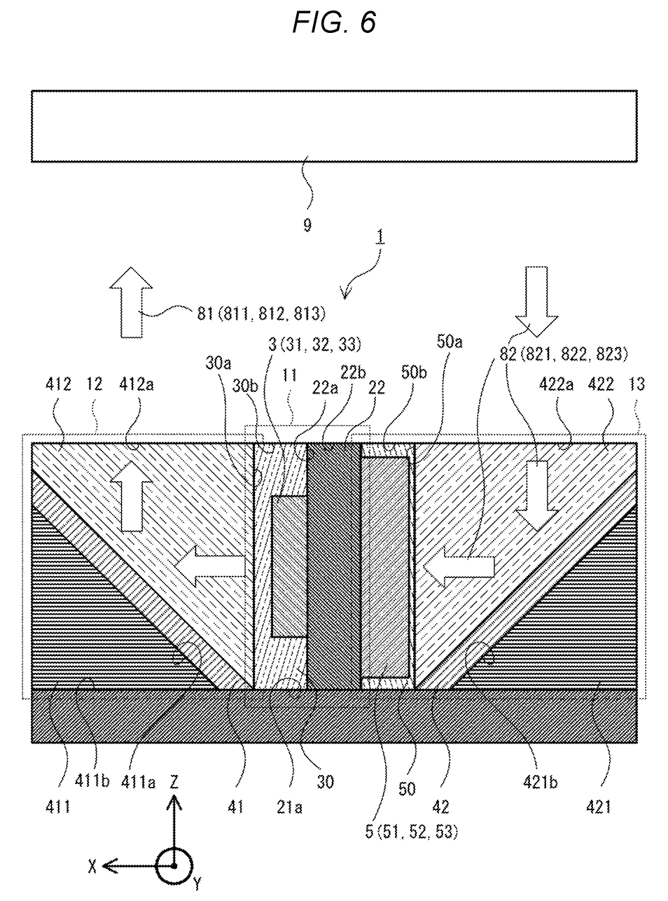

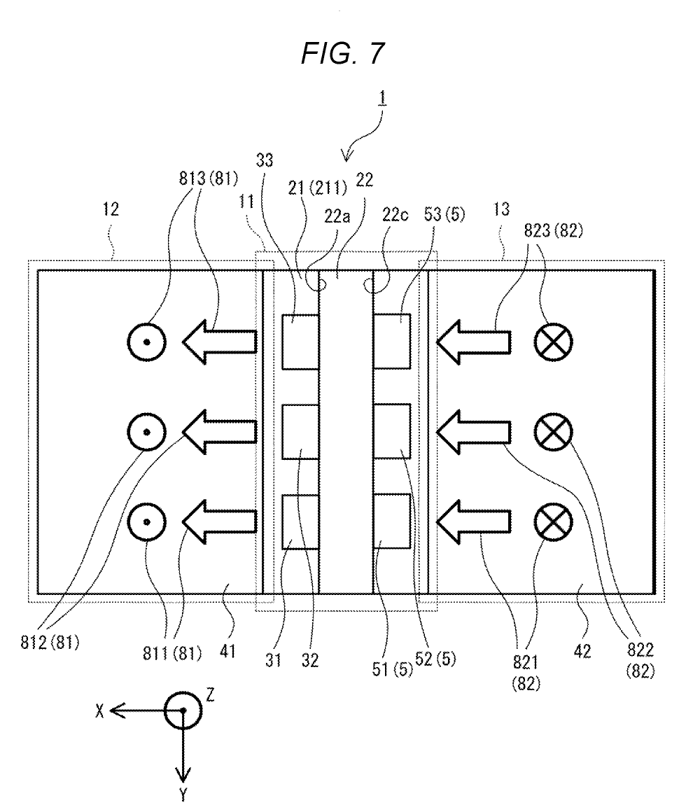

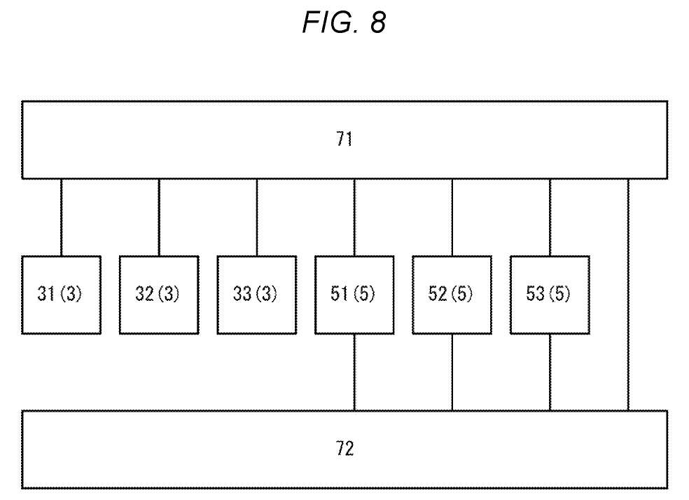

FIG. 6 is a cross-sectional view of an example of the configuration of a living body detection apparatus 1 according to a third embodiment as viewed from the first direction (Y direction). FIG. 7 is a plan view of the example of the configuration of the living body detection apparatus 1 shown in FIG. 6 as viewed from the second direction (Z direction) perpendicular to the first direction. FIG. 8 is a block circuit diagram schematically showing an example of the electrical coupling in the living body detection apparatus 1 according to the third embodiment. The living body detection apparatus 1 according to the third embodiment is the living body detection apparatus 1 according to the second embodiment to which the following modifications are added, as shown in FIGS. 6, 7, and 8. Note in the third embodiment that portions common to those in the second embodiment will not be described in detail in some cases.

The configurations of the second unit 12 and the third unit 13 according to the third embodiment are the same as those in the second embodiment.

In the first unit 11, a first light receiver 51, a second light receiver 52, and a third light receiver 53 are provided as the light receiver 5, as shown in FIGS. 6 and 7. The first light emitter 31 and the first light receiver 51 may overlap with each other in a plan view as viewed from the direction in which the first light emitter 31 outputs the first output light 811 (+X direction). Similarly, the second light emitter 32 and the second light receiver 52 may overlap with each other in a plan view as viewed from the direction in which the second light emitter 32 outputs the second output light 812 (+X direction). The third light emitter 33 and the third light receiver 53 may overlap with each other in a plan view as viewed from the direction in which the third light emitter 33 outputs the third output light 813 (+X direction).

In the first unit 11, the first light receiver 51 may be configured to receive and detect the first reflected light 821 derived from the first output light 811 having the first color, but not to detect the second reflected light 822 derived from the second output light 812 having the second color or the third reflected light 823 derived from the third output light 813 having the third color even when the first light receiver 51 receives the second reflected light 822 and the third reflected light 823, as shown in FIGS. 6 and 7. Similarly, the second light receiver 52 may be configured to receive and detect the second reflected light 822 derived from the second output light 812 having the second color, but not to detect the first reflected light 821 derived from the first output light 811 having the first color or the third reflected light 823 derived from the third output light 813 having the third color even when the second light receiver 52 receives the first reflected light 821 and the third reflected light 823. The third light receiver 53 may be configured to receive and detect the third reflected light 823 derived from the third output light 813 having the third color, but not to detect the first reflected light 821 derived from the first output light 811 having the first color and the second reflected light 822 derived from the second output light 812 having the second color even when the third light receiver 53 receives the first reflected light 821 and the second reflected light 822.

The detector 72 according to the third embodiment is electrically coupled to each of the first light receiver 51, the second light receiver 52, and the third light receiver 53 provided in the light receiver 5, as shown in FIG. 8. As a result, the detector 72 can separately receive a first electric signal, a second electric signal, and a third electric signal generated based on the first reflected light 821, the second reflected light 822, and the third reflected light 823 received by the first light receiver 51, the second light receiver 52, and the third light receiver 53, respectively. As a result, in the third embodiment, living body information on the detection target living body 9 can be accurately detected without performing the intermittent driving or the coordinated operation of the light emitter 3 and/or the light receiver 5, unlike in the first and second embodiments. Performing no intermittent driving or coordinated operation of the light emitter 3 and/or the light receiver 5 further allows reduction in the power consumed by the living body detection apparatus 1.

Note that the controller 71 according to the third embodiment can also separately control the operations of the first light emitter 31, the second light emitter 32, and the third light emitter 33 provided in the light emitter 3, the operations of the first light receiver 51, the second light receiver 52, and the third light receiver 53 provided in the light receiver 5, and the operation of the detector 72. The intermittent driving, the coordinated operation, and the like of the light emitter 3 and the light receiver 5 can therefore be achieved, as in the first and second embodiments.

### Summary of Present Disclosure

The present disclosure will be summarized below as additional remarks.

### Additional Remark 1

A living body detection apparatus including:

a light emitter configured to emit first light;

a first reflector configured to reflect the first light in a first direction;

a light receiver configured to receive second light that is the first light reflected from a detection target living body when the detection target living body is present in the first direction when viewed from the first reflector;

a first substrate disposed between the light emitter and the light receiver and configured to block the first light; and

a detector configured to detect living body information on the detection target living body based on the second light,

wherein the light emitter is disposed between the light receiver and the first reflector in a plan view viewed from the detection target living body, and

the light emitter emits the first light toward the first reflector.

The configuration that prevents the first light emitted by the light emitter from directly reaching the light receiver allows accurate detection of living body information on the detection target living body.

The configuration in which the first substrate is disposed between the light emitter and the light receiver allows reduction in the size of the living body detection apparatus.

### Additional Remark 2

The living body detection apparatus according to Additional Remark 1, wherein

an output portion via which the light emitter emits the first light is provided on a side opposite the light receiver in the plan view.

The configuration that prevents the first light emitted by the light emitter from directly reaching the light receiver allows accurate detection of living body information on the detection target living body.

### Additional Remark 3

The living body detection apparatus according to Additional Remark 1, wherein

a direction in which the light emitter emits the first light is a second direction toward the first reflector when viewed from the light emitter.

The configuration that prevents the first light emitted by the light emitter from directly reaching the light receiver allows accurate detection of living body information on the detection target living body.

### Additional Remark 4

The living body detection apparatus according to Additional Remark 1, wherein

the light emitter includes

a first light emitter configured to emit light that is included in the first light and belongs to a first wavelength band,

a second light emitter configured to emit light that is included in the first light and belongs to a second wavelength band, and

a third light emitter configured to emit light that is included in the first light and belongs to a third wavelength band.

Using multiple types of light that belong to different wavelength bands allows accurate detection of different types of living body information, such as pulse measurement and blood oxygen saturation concentration measurement.

### Additional Remark 5

The living body detection apparatus according to Additional Remark 1, further including

a second substrate extending along a first plane and configured to support the first reflector,

wherein the first substrate extends along a second plane that intersects with the first plane and is configured to support the light emitter and the light receiver,

the light emitter is provided at a first surface of the second substrate, and

the light receiver is provided at a second surface of the second substrate that is a surface facing the first surface.

The configuration in which the first substrate is disposed between the light emitter and the light receiver allows reduction in the size of the living body detection apparatus.

### Additional Remark 6

The living body detection apparatus according to Additional Remark 5, wherein

the light emitter includes

a first light emitter configured to emit light that is included in the first light and belongs to a first wavelength band,

a second light emitter configured to emit light that is included in the first light and belongs to a second wavelength band, and

a third light emitter configured to emit light that is included in the first light and belongs to a third wavelength band.

Using multiple types of light that belong to different wavelength bands allows accurate detection of different types of living body information, such as pulse measurement and blood oxygen saturation concentration measurement.

### Additional Remark 7

The living body detection apparatus according to Additional Remark 6, wherein

the light receiver includes

a first light receiver configured to receive light that is included in the second light and belongs to the first wavelength band,

a second light receiver configured to receive light that is included in the second light and belongs to the second wavelength band, and

a third light receiver configured to receive light that is included in the second light and belongs to the third wavelength band.

Living body information on the detection target living body can be accurately detected without performing intermittent driving or coordinated operation of the light emitter and/or the light receiver.

### Additional Remark 8

The living body detection apparatus according to Additional Remark 7, wherein

in a plan view as viewed from a first direction in which the light emitter emits the first light,

the first light emitter and the first light receiver overlap with each other,

the second light emitter and the second light receiver overlap with each other, and

the third light emitter and the third light receiver overlap with each other.

### Additional Remark 9

The living body detection apparatus according to Additional Remark 7, further including

a controller configured to control the light emitter and the light receiver,

wherein the controller is configured to perform

first control of causing the first light emitter and the first light receiver to operate and causing the second light emitter and the second light receiver not to operate during a first period, and

second control of causing the first light emitter and the first light receiver not to operate and causing the second light emitter and the second light receiver to operate during a second period.

Suppressing the emission of light that becomes noise allows accurate detection of living body information on the detection target living body.

### Additional Remark 10

The living body detection apparatus according to Additional Remark 1, wherein

the light emitter includes a light-emitting element configured to emit the first light in the form of surface emission.

### Additional Remark 11

The living body detection apparatus according to Additional Remark 1, further including:

a first unit including the first reflector, a first support configured to support a first surface of the first reflector, and a first protector configured to protect a second surface of the first reflector, the second surface facing the first surface;

a second unit including the first substrate and the light emitter; and

a second substrate configured to support the first unit and the second unit.

### Additional Remark 12

The living body detection apparatus according to Additional Remark 11, further including

a third unit including the light receiver, a second protector configured to protect the light receiver, and a third substrate configured to support the light receiver,

wherein the second substrate is configured to further support the third unit.

### Additional Remark 13

The living body detection apparatus according to Additional Remark 11, wherein

in the second unit, the light emitter is mounted on a first surface of the second substrate, and the light receiver is mounted on a second surface facing the first surface,

the second unit further includes a third unit including a second reflector, a second support configured to support a first surface of the second reflector, and a second protector configured to protect a second surface of the second reflector that is a surface facing the first surface, and

the second substrate is configured to further support the third unit.

The living body detection apparatus is produced by joining the multiple units, which are produced independently of each other, to each other on the same substrate. As a result, reduction in production difficulty, improvement in production yield, reduction in production cost, and the like are expected.

The disclosure made by the present discloser has been specifically described based on the embodiments. The present disclosure is not limited to the embodiments, and it goes without saying that various changes can be made thereto without departing from the key points of the present disclosure. In addition, the features described in the embodiments can be freely combined with each other as long as the combined features do not technically contradict each other.

What is claimed is:

1. A living body detection apparatus comprising:
a light emitter configured to emit first light;
a first reflector configured to reflect the first light toward a detection target living body;
a light receiver configured to receive second light output from the detection target living body on which the first light is incident;
a detector configured to detect living body information on the detection target living body based on the second light, and
a first substrate disposed between the light emitter and the light receiver in a plan view viewed from the detection target living body;
wherein the light emitter is disposed between the light receiver and the first reflector in the plan view.

2. The living body detection apparatus according to claim 1, wherein
the light emitter has an output portion via which the light emitter emits the first light, and
the output portion is provided on a side opposite the light receiver in the plan view.

3. The living body detection apparatus according to claim 1, wherein
the light emitter is configured to emit the first light toward the first reflector.

4. The living body detection apparatus according to claim 1, wherein
the light emitter includes
a first light emitter configured to emit light that is included in the first light and belongs to a first wavelength band,
a second light emitter configured to emit light that is included in the first light and belongs to a second wavelength band, and
a third light emitter configured to emit light that is included in the first light and belongs to a third wavelength band.

5. The living body detection apparatus according to claim 1, further comprising
a second substrate extending along a first plane and configured to support the first reflector,
wherein the first substrate extends along a second plane that intersects with the first plane and is configured to support the light emitter and the light receiver,
the light emitter is provided at a first surface of the first substrate, and
the light receiver is provided at a second surface of the first substrate that is a surface facing the first surface.

6. The living body detection apparatus according to claim 5, wherein
the light emitter includes
a first light emitter configured to emit light that is included in the first light and belongs to a first wavelength band,
a second light emitter configured to emit light that is included in the first light and belongs to a second wavelength band, and
a third light emitter configured to emit light that is included in the first light and belongs to a third wavelength band.

7. The living body detection apparatus according to claim 6, wherein
the light receiver includes
a first light receiver configured to receive light that is included in the second light and belongs to the first wavelength band,
a second light receiver configured to receive light that is included in the second light and belongs to the second wavelength band, and
a third light receiver configured to receive light that is included in the second light and belongs to the third wavelength band.

8. The living body detection apparatus according to claim 7, wherein
in a plan view as viewed from a direction in which the light emitter emits the first light,
the first light emitter and the first light receiver overlap with each other,
the second light emitter and the second light receiver overlap with each other, and
the third light emitter and the third light receiver overlap with each other.

9. The living body detection apparatus according to claim 7, further comprising
a controller configured to control the light emitter and the light receiver,
wherein the controller is configured to perform
first control of causing the first light emitter and the first light receiver to operate and causing the second light emitter and the second light receiver not to operate during a first period, and
second control of causing the first light emitter and the first light receiver not to operate and causing the second light emitter and the second light receiver to operate during a second period.

10. The living body detection apparatus according to claim 1, wherein
the light emitter includes a light-emitting element configured to emit the first light in the form of surface emission.

11. The living body detection apparatus according to claim 1, further comprising:
a first unit including the first reflector, a first support configured to support a first surface of the first reflector, and a first protector configured to protect a second surface of the first reflector, the second surface facing the first surface;
a second unit including the first substrate and the light emitter; and
a second substrate configured to support the first unit and the second unit.

12. The living body detection apparatus according to claim 11, further comprising
a third unit including the light receiver, a second protector configured to protect the light receiver, and a third substrate configured to support the light receiver,
wherein the second substrate is configured to further support the third unit.

13. The living body detection apparatus according to claim 11, wherein
in the second unit, the light emitter is mounted on a first surface of the second substrate, and the light receiver is mounted on a second surface facing the first surface,
the second unit further comprises a third unit including a second reflector, a second support configured to support a first surface of the second reflector, and a second protector configured to protect a second surface of the second reflector that is a surface facing the first surface, and
the second substrate is configured to further support the third unit.
